(19) United States
(12) Patent Application Publication
(10) Pub. No.: US 2009/0312284 A1
(43) Pub. Date: Dec. 17, 2009

(54) HISTONE DEACETYLASE INHIBITORS WITH COMBINED ACTIVITY ON CLASS-I AND CLASS-IIB HISTONE DEACETYLASES IN COMBINATION WITH PROTEASOME INHIBITORS

(76) Inventors: Janine Arts, Breda (NL); Peter Willem Jan Hellemans, Bonheiden (BE); Michel Marie Francois Janicot, Mol (BE); Martin John Page, Schilde (BE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/441,218

(22) PCT Filed: Sep. 11, 2007
(86) PCT No.: PCT/EP07/59518

§ 371 Date: Mar. 13, 2009

Related U.S. Application Data
(60) Provisional application No. 60/915,895, filed on May 3, 2007.

(30) Foreign Application Priority Data
Sep. 15, 2006 (EP) 06120726.2

(51) Int. Cl. A61K 31/69; A61K 31/506; C12Q 1/25; A61P 35/00

(52) U.S. Cl. 514/64; 514/275; 435/4

(57) ABSTRACT

The present invention is concerned with combinations of a proteasome inhibitor and a histone deacetylase inhibitor with combined activity on class-I and class-IIb histone deacetylases, for inhibiting the growth of tumor cells, useful in the treatment of cancer.

The present invention relates to histone deacetylase (HDAC) inhibitors with combined activity on class-I and class-II histone deacetylases. It relates to combinations and compositions comprising them, as well as to their use, as a medicine, for instance as a medicine to inhibit hematopoietic tumors such as lymphomas and leukemias.

The family of HDAC enzymes has been named after their first substrate identified, i.e., the nuclear histone proteins. Histone proteins (H2A, H2B, H3 and H4) form an octamer complex, around which the DNA helix is wrapped in order to establish a condensed chromatin structure. The acetylation status of histones is in dynamic equilibrium governed by histone acetyl transferases (HATs), which acetylate and HDACs which are responsible for the deacetylation of histone tails. Inhibition of the HDAC enzyme promotes the acetylation of the nucleosome histone tails, favoring a more transcriptionally competent chromatin structure, which in turn leads to altered expression of genes involved in cellular processes such as cell proliferation, apoptosis and differentiation. In recent years, a growing number of additional non-histone HDAC substrates have been identified.

Disregulated and constant HDAC recruitment in conjunction with oncogenic transcription factors to the chromatin is observed in specific forms of leukemia and lymphoma, such as acute promyelocytic leukemia (APL), non-Hodgkin's lymphoma and acute myeloid leukemia (AML). Upregulation of HDAC1 at the protein level was observed in prostate cancer cells, as the disease progresses from malignant lesions and well-differentiated androgen-responsive prostate adenocarcinoma towards the phenotypically de-differentiated androgen insensitive prostate cancer. In addition, increased HDAC2 expression is found in the majority of human colon cancer explants which is triggered by the loss of the tumor suppressor adenomatosis polyposis coli (APC).

In agreement with the HDAC/HAT activity equilibrium in cancer, HDAC inhibitors have been shown to induce cell-cycle arrest, terminal differentiation and/or apoptosis in a broad spectrum of human tumor cell lines in vitro, to inhibit angiogenesis and to exhibit in vivo antitumor activity in human xenograft models in nude mice.

The HDAC family of enzymes are commonly divided into 3 classes: i.e., classes I, II and III. Only Classes I and II have been predominantly implied to mediate the effects of HDAC inhibitors currently in clinical development.

The class-I group HDACs, which consists of HDAC family members 1-3 and 8 have been shown to be crucial for tumor cell proliferation.

Among the wide variety of transcription factors that utilize class-I HDACs to silence specific promotors, the best known example is the class of nuclear hormone receptors, which only bind HDAC3 in absence of their ligand, and thus maintain a state of transcriptional silencing. The complex is dissociated in a ligand-dependent manner, e.g., by retinoids, estrogens, androgens, et cetera, resulting in gene expression and differentiation. Another key example is the HDAC1-dependent silencing of the cyclin-dependent kinase inhibitor p21^{waf1,cip1}. The crucial role of p21^{waf1,cip1} induction in the antiproliferative effects of HDAC inhibitors was demonstrated by studies showing a 6-fold increase in resistance to the HDAC inhibitor trichostatin A (TSA) in p21^{waf1,cip1} deficient cells as compared to the parental HCT-116 cells. In addition, unlike genuine tumor suppressor genes, p21^{waf1,cip1} is ubiquitously present in tumor cells, and induced by HDAC inhibitors.

Histones are not the only substrates of the class-I HDACs. For example, HDACs 1-3 deacetylase the tumor suppressor p53, which as a consequence gets ubiquitinated and degraded. Since p53 is a potent tumor suppressor, including cell cycle arrest and apoptosis, maintaining low levels of this protein is key for allowing survival and uncontrolled proliferation of tumor cells.

The class-II HDACs can be divided into 2 subclasses: class-IIa containing HDACs 4, 5, 7, 9 and the HDAC 9 splice variant MITR. Class-IIb comprises HDAC6 and HDAC 10, which both have duplicated HDAC domains. Class-IIa HDACs do not possess intrinsic histone deacetylase activity but regulate gene expression by functioning as the bridging factors since they associate both with class-1 HDAC complexes and with transcription factor/DNA complexes.

HDAC6, a member of class-IIb, has received attention due to its identification as a Hsp90 deacetylase. The HDAC inhibitors LAQ824 and LBH589 have been demonstrated to induce the deacetylation of Hsp90 while trapoxin and sodium butyrate do not. Hsp90 deactylase results in degradation of Hsp90 associated pro-survival and pro-proliferative client proteins. Key examples include Her-2, Bcr-Abl, glucocorticoid receptor, mutant FLT-3, c-Raf and Akt. In addition to Hsp90, HDAC6 also mediates tubulin deacetylation which results in microtubule destabilization under stressed conditions.

The biological role of HDAC6 was further confirmed by the fact that a specific small molecule inhibitor of HDAC6, tubacin, caused α-tubulin hyperacetylation and decreased cell motility without affecting cell cycle progression. Tubacin, which inhibits only the α-tubulin deacetylase domain of HDAC6, causes only a minimal increase in HSP90 acetylation.

In agreement, HDAC6 was found to be key for the estradiol-stimulated cell migration of MCF-7 breast carcinoma cells.

Finally, HDAC6 plays a crucial role in the cellular management of misfolded proteins and clearing these from the cytoplasm.

Due to the large number of cell cycle regulatory proteins regulated by HDACs at the level of either their expression or activity, the antiproliferative effect of HDAC inhibitors cannot be linked to a single mechanism of action. HDAC inhibition holds particular promise in anticancer therapy, where the concerted effects on multiple pathways involved in growth inhibition, differentiation and apoptosis may prove advantageous in the treatment of a heterogeneous pathology such as tumor formation and growth.

Over the years, it has become evident that HDACs do not only play a key role in carcinogenesis, but also in a number of non-malignant differentiation processes. This is most apparent for the class-IIa 4, 5, 7 and 9. For example, HDAC7 has been suggested to play a critical role in the thymic maturation of T-cells, while HDAC4 has been implicated in the regulation of chondrocyte hypertrophy and endochondral bone formation. Most concerns, however, have focused around the role of the class-IIa HDACs in muscle differentiation. HDACs 4, 5, 7 and 9 all suppress the differentiation of myocytes (muscle cells) as a consequence of being transcriptional co-repressors of myocyte enhancer factor 2 (MEF2).

The most common toxicity seen with HDAC inhibitors is myelosuppresion of mild to moderate degree. In addition, nausea/vomiting, fatigue and diarrhea feature as adverse effects in many clinical trials.

EP 1485365 published on 18 Sep. 2003 discloses amongst others the HDAC inhibitor R306465.

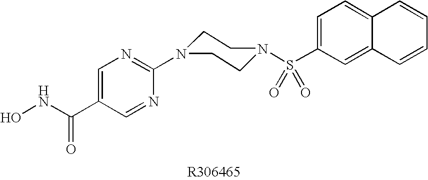

WO 2006/010750 published on 2 Feb. 2006 describes the preparation, formulation and pharmaceutical properties of compounds with the following Markush formula.

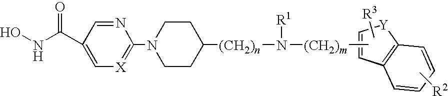

the N-oxide forms, the pharmaceutically acceptable addition salts and the stereo-chemically isomeric forms thereof,
wherein n, m, R¹, R², R³, X and Y have the meanings as defined in said specification.

The potential for HDAC inhibitor therapy however goes beyond single agent use. The molecular pathways affected by HDAC inhibitors make it a promising candidate for combinatorial studies.

There is a need for inhibitors with combined effects on class-I and class-IIb HDACs that can offer clinical advantages considering efficacy and/or toxicity. either alone or in combinations with other therapeutic agents,

Also proteasome inhibition represents an important recently developed strategy in cancer therapy. The proteasome is a multi-enzyme complex present in all cells which play a role in degradation of proteins involved in regulation of the cell cycle. A number of key regulatory proteins, including p53, cyclins and the cyclin-dependent kinase p21^{waf1,cip1} are temporally degraded during the cell cycle by the ubiquitin-proteasome pathway. The ordered degradation of these proteins is required for the cell to progress through the cell cycle and to undergo mitosis. Furthermore, the ubiquitin-proteasome pathway is required for transcriptional regulation.

EP788360, EP1312609, EP1627880, U.S. Pat. No. 6,066,730 and U.S. Pat. No. 6,083,903 discloses peptide boronic ester and acid compounds useful as proteasome inhibitors. One of the compounds N-pyrazinecarbonyl-L-phenylalanine-L-leucineboronic acid (PS-341, now known as bortezomib or Velcade (Millenium)) has antitumor activity in human tumor xenograft models and has received approval for the treatment of patients having relapsed refractory multiple myeloma, and is presently undergoing clinical trials in additional indications, including additional haematological cancers as well as solid tumors. Bortezomib induces cell death by causing a buildup of misfolded and otherwise damaged proteins thereby activating the mitochondrial pathway of apoptosis, for example via Bax- or reactive oxygen species dependent mechanisms.

Bortezomib causes the sequestration of ubiquitin-conjugated proteins into structures termed aggresomes. Aggresomes seem to participate in a cytoprotective response that is activated in response to proteasome inhibition perhaps by shuttling ubiquitylated proteins to lysosomes for degradation.

Bortezomib-induced aggresome formation could be disrupted using the HDAC inhibitor SAHA (suberoylanilide hydroxamic acid). SAHA also demonstrates synergistic effects on apoptosis in vitro and in an orthotopic pancreatic cancer xenograft model in vivo (Cancer Research 2006; 66: (7) 3773-3781).

Another HDAC inhibitor LAQ824 also demonstrate synergistic levels of cell death with bortezomib (Journal of Biological Chemistry 2005; 280: (29) 26729-26734).

The synergistic effect of SAHA and LAQ824 with bortezomib have been related to their HDAC6 inhibitory activity.

There is a further need to increase the inhibitory efficacy of proteasome inhibitors against tumor growth and also to lower dosages of such agents to reduce the potential of adverse toxic side effects to the patient.

At the moment robust data of correlation of the degree of acetylation with tumor response is not available. Quick, simple and easily reproducible methods of quantifying the degree of acetylation of histone and non-histone substrates caused by the below described HDAC inhibitors or combinations comprising said HDAC inhibitors will be crucial to their future.

It is an object of the invention to provide HDAC inhibitors and therapeutic combinations of a proteasome inhibitor and HDAC inhibitors of the type described below which can have robust and characteristic acetylation effects, inhibition of both class-I and class-IIb HDACs, advantageous inhibitory effect against tumor cell growth, and less undesired side effects.

According to the invention therefore we provide a combination of a proteasome inhibitor and a HDAC inhibitor of formula (I)

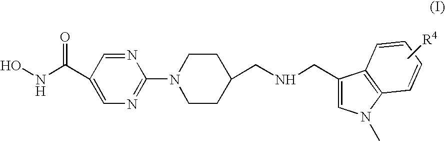

the pharmaceutically acceptable acid or base addition salts and the stereochemically isomeric forms thereof, wherein

R⁴ is selected from hydrogen or halo.

Interesting compounds are those compounds of formula (I) wherein R⁴ is fluor.

More interesting compounds are those compounds of formula (I) wherein R⁴ is in the 4 or the 7 position of the indole.

Preferred compounds of formula (I) are compound No. 1a, compound No. 30 and compound No. 39 corresponding to the numbering as indicated in WO 2006/010750.

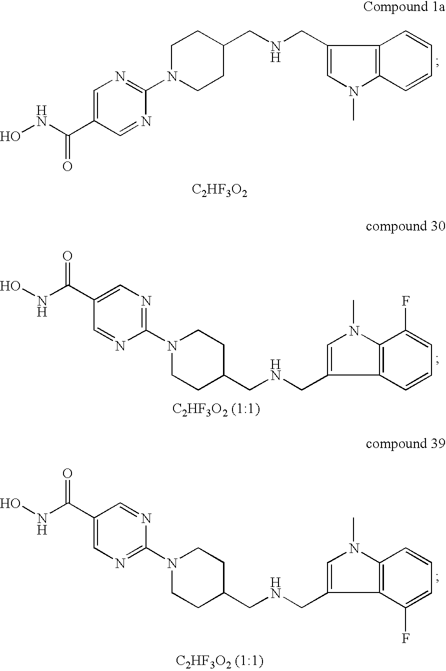

Other preferred compound of formula (I) are the compounds wherein R⁴ is hydrogen.

The most preferred compound is compound No. 1a (JNJ26481585)

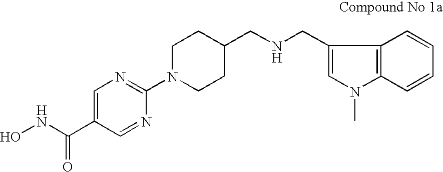

or a pharmaceutically acceptable addition salt thereof.

Lines drawn into the bicyclic ring systems from substituents indicate that the bonds may be attached to any of the suitable ring atoms of the bicyclic ring system.

As used in the foregoing definitions and hereinafter, halo is generic to fluoro, chloro, bromo and iodo.

As used herein, the terms “histone deacetylase” and “HDAC” are intended to refer to any one of a family of enzymes that remove acetyl groups from the ε-amino groups of lysine residues at the N-terminus of a histone. Unless otherwise indicated by context, the term “histone” is meant to refer to any histone protein, including H1, H2A, H2B, H3, H4, and H5, from any species. Human HDAC proteins or gene products, include, but are not limited to, HDAC-1, HDAC-2, HDAC-3, HDAC-4, HDAC-5, HDAC-6, HDAC-7, HDAC-8, HDAC-9, HDAC-10 and HDAC-11. The histone deacetylase can also be derived from a protozoal or fungal source.

The term “histone deacetylase inhibitor” or “inhibitor of histone deacetylase” is used to identify a compound, which is capable of interacting with a histone deacetylase and inhibiting its activity, more particularly its enzymatic activity. Inhibiting histone deacetylase enzymatic activity means reducing the ability of a histone deacetylase to remove an acetyl group from a histone or another protein substrate. Preferably, such inhibition is specific, i.e. the histone deacetylase inhibitor reduces the ability of a histone deacetylase to remove an acetyl group from a histone or another protein substrate at a concentration that is lower than the concentration of the inhibitor that is required to produce some other, unrelated biological effect.

The term “HDAC inhibitors with combined activity on class-I and class-IIb HDACs” or “inhibition of class-I and class-IIb HDACs” is used to identify compounds which reduce the enzymatic activity of both a class-I HDAC family member (HDAC1-3 or 8) and a class IIb HDAC family member (HDAC 6 or 10) at a concentration that is lower than the concentration of the inhibitor that is required to produce inhibition of other classes of HDAC enzymes such as e.g. class-IIa or at a concentration that is lower than the concentration of the inhibitor that is required to produce inhibition of some other related biological effect.

As used herein, the terms “proteasome” and “ubiquitin-protesome system (UPS)” are intended to refer to any one of the structures and functions of all components in the UPS which include, but are not limited to:
a) the ubiquitin (Ub) and ubiquitin-like proteins (Ulp); f.e. SUMO, NEDD8, ISG15 and the like,
b) ubiquitin monomers, K48-linked polyubiquitin chains, K63-linked polyubiquitin chains and the like,
c) the E1 ubiquitin-activating enzymes; f.e. E1^{Ub}, E1^{SUMO}, E1^{NEDD8}, E1^{ISG15} and the like,
d) subunits of the E1 ubiquitin-activating enzymes; f.e. APPBP1, UBA3, SAE1, SAE2 and the like,
e) the E2 ubiquitin-conjugating enzymes; f.e. UBC9, UBC12, UBC8 and the like,
f) the E3 ubiquitin ligases; f.e. RING-finger E3s, simple RING-finger E3s, cullin-based RING-finger E3 s, RBX1-/RBX2-dependent E3 s, HECT-domain E3s, U-box E3s, and the like,
g) the SCF (SKP1-Cullin1-F-box) E3 ubiquitin ligase complex, f.e. SCF^{SKP2}, SCF^{B-TRCP}, SCF^{FBW7} and the like,
h) the cullins, f.e. CUL1, CUL2, CUL3, CUL4, CUL5 and the like,
i) the F-box proteins f.e. SKP2, B-TRCP proteins, FBW proteins and the like,
j) other substrate specific adaptors, f.e. BTB proteins, SOCS-box proteins, DDB1/2, VHL and the like,
k) the proteasome, its components and the like,
l) the metalloisopeptidase RPN11, a subunit of the proteasome lid, that de-ubiquitilates UPS targets prior to their destruction, and the like,
m) the metalloisopeptidase CSN5, a subunit of the COP9-signalosome complex, that is responsible for removing NEDD8 from cullins, and the like,
n) the activation step, by a E1 ubiquitin-activating enzyme, in which the Ub/Ulp first becomes adenylated on its C-terminal glycine residue and then becomes charged as a thiol ester, again at its C-terminus,
o) the transfer of the Ub/Ulp from a E I ubiquitin-activating enzymes to a E2 ubiquitin-conjugating enzyme,
p) ubiquitin-conjugate recognition,
q) transfer and binding of the substrate-ubiquitin complex to the proteasome,
r) ubiquitin removal, or
s) substrate degradation.

The term “proteasome inhibitor” and “inhibitor of the ubiquitin-proteasome system” is used to identify a compound, which is capable of interacting with one of the normal, altered, hyper-active or overexpressed components in the UPS and inhibiting its activity, more particularly its enzymatic activity. Inhibiting UPS enzymatic activity means reducing the ability of a UPS component to perform its activity. Preferably, such inhibition is specific, i.e. the proteasome inhibitor reduces the activity of a component of the UPS at a concentration that is lower than the concentration of the inhibitor that is required to produce some other, unrelated biological effect. Inhibitors of the activity of a UPS component includes, but are not limited to:
a) inhibitors of Ub or Ulp adenylation by blocking access of Ub/Ulp to the adenylate site or by blocking access of ATP; f.e. imatinib (Gleevec; Novartis) and the like,
b) disruptors of the interaction of the E3 or E3-complex with E2,
c) interrupters of the interaction between the substrate and the substrate interaction domain on the E3 or E3-complex, such as blocking the interaction between p53 (the substrate) and MDM2 (the RING-finger E3) f.e. nutlins (by binding to MDM2), RITA (by binding to the N terminus of p53) and the like,
d) interrupters of the E3 ligase complex,
e) artificially recruiters of substrates to the ubiquitin ligases, f.e. protacs and the like,
f) inhibitors of the proteasome and its components f.e. bortezomib, carfilzomib, NPI-0052, Bsc2118 and the like,
g) inhibitors of the ubiquitin/Ulp removal such as inhibitors of the metalloisopeptidases RPN11 and CSN5, or
h) modifying the polyubiquitin chain f.e. ubistatins and the like.

The pharmaceutically acceptable acid addition salts as mentioned hereinabove are meant to comprise the therapeutically active non-toxic acid addition salt forms which the compounds of formula (I) are able to form. The compounds of formula (I) which have basic properties can be converted in their pharmaceutically acceptable acid addition salts by treating said base form with an appropriate acid. Appropriate acids comprise, for example, inorganic acids such as hydrohalic acids, e.g. hydrochloric or hydrobromic acid; sulfuric; nitric; phosphoric and the like acids; or organic acids such as, for example, acetic, trifluoroacetic, propanoic, hydroxyacetic, lactic, pyruvic, oxalic, malonic, succinic (i.e. butanedioic acid), maleic, fumaric, malic, tartaric, citric, methanesulfonic, ethanesulfonic, benzenesulfonic, p-toluenesulfonic, cyclamic, salicylic, p-amino-salicylic, pamoic and the like acids.

The compounds of formulae (I) which have acidic properties may be converted in their pharmaceutically acceptable base addition salts by treating said acid form with a suitable organic or inorganic base. Appropriate base salt forms comprise, for example, the ammonium salts, the alkali and earth alkaline metal salts, e.g. the lithium, sodium, potassium, magnesium, calcium salts and the like, salts with organic bases, e.g. the benzathine, N-methyl-D-glucamine, hydrabamine salts, and salts with amino acids such as, for example, arginine, lysine and the like.

The terms acid or base addition salt also comprise the hydrates and the solvent addition forms which the compounds of formulae (I) are able to form. Examples of such forms are e.g. hydrates, alcoholates and the like.

The term stereochemically isomeric forms of compounds of formulae (I) as used hereinbefore, defines all possible compounds made up of the same atoms bonded by the same sequence of bonds but having different three-dimensional structures which are not interchangeable, which the compounds of formulae (I) may possess. Unless otherwise mentioned or indicated, the chemical designation of a compound encompasses the mixture of all possible stereochemically isomeric forms which said compound may possess. Said mixture may contain all

diastereomers and/or enantiomers of the basic molecular structure of said compound. All stereochemically isomeric forms of the compounds of formulae (I) both in pure form or in admixture with each other are intended to be embraced within the scope of the present invention.

Some of the compounds of formula (I) may also exist in their tautomeric forms. Such forms although not explicitly indicated in the above formula are intended to be included within the scope of the present invention.

Whenever used hereinafter, the term “compounds of formula (I) is meant to include also the pharmaceutically acceptable acid or base addition salts and all stereoisomeric forms.

A particularly preferred proteasome inhibitor for use in accordance with the invention is bortezomib. Bortezomib is commercially available from Millennium under the trade name Velcade and may be prepared for example as described in EP788360, EP1312609, EP1627880, U.S. Pat. No. 6,066,730 and U.S. Pat. No. 6,083,903 or by processes analogous thereto.

The present invention also relates to combinations according to the invention for use in medical therapy for example for inhibiting the growth of tumor cells.

The present invention also relates to the use of combinations according to the invention for the preparation of a pharmaceutical composition for inhibiting the growth of tumor cells.

The present invention also relates to a method of inhibiting the growth of tumor cells in a human subject which comprises administering to the subject an effective amount of a combination according to the invention.

This invention further provides a method for inhibiting the abnormal growth of cells, including transformed cells, by administering an effective amount of a combination according to the invention. Abnormal growth of cells refers to cell growth independent of normal regulatory mechanisms (e.g. loss of contact inhibition). This includes the inhibition of tumour growth both directly by causing growth arrest, terminal differentiation and/or apoptosis of cancer cells, and indirectly, by inhibiting migration, invasion and survival of tumor cells or neovascularization of tumors.

This invention also provides a method for inhibiting tumor growth by administering an effective amount of a combination according to the present invention, to a subject, e.g. a mammal (and more particularly a human) in need of such treatment. In particular, this invention provides a method for inhibiting the growth of tumors by the administration of an effective amount of the combination according to the present invention. The present invention is particularly applicable to the treatment of pancreatic cancer, hematopoietic tumors of lymphoid lineage e.g. acute lymphoblastic leukemia, acute myelogenous leukemia, acute promyelocytic leukemia, acute myeloid leukemia, acute monocytic leukemia, lymphoma, chronic B cell leukemia, chronic myeloid leukemia, chronic myeloid leukemia in blast crisis, Burkitt's lymphoma, multiple myeloma, non-small-cell lung cancer, small-cell lung cancer, non-Hodgkin's lymphoma, melanoma, prostate cancer, breast cancer and colon cancer. Examples of other tumors which may be inhibited include, but are not limited to, thyroid follicular cancer, myelodysplastic syndrome (MDS), tumors of mesenchymal origin (e.g. fibrosarcomas and rhabdomyosarcomas), teratocarcinomas, neuroblastomas, gliomas, benign tumor of the skin (e.g. keratoacanthomas), kidney carcinoma, ovary carcinoma, bladder carcinoma and epidermal carcinoma.

This invention also provides a method for the treatment of acute lymphoblastic leukemia, acute myelogenous leukemia, acute promyelocytic leukemia, acute myeloid leukemia, acute monocytic leukemia, lymphoma, chronic B cell leukemia, chronic myeloid leukemia, chronic myeloid leukemia in blast crisis, Burkitt's lymphoma and multiple myeloma by administering an effective amount of a histone deactylase inhibitor of formula (I), to a subject, e.g. a mammal (and more particularly a human) in need of such treatment.

This invention also provides a method for the treatment of drug resistant tumors, such as but not limited to hematopoietic tumors of lymphoid lineage e.g. drug resistant acute lymphoblastic leukemia, drug resistant acute myelogenous leukemia, drug resistant acute promyelocytic leukemia, drug resistant acute myeloid leukemia, drug resistant acute monocytic leukemia, drug resistant lymphoma, drug resistant chronic B cell leukemia, drug resistant chronic myeloid leukemia, drug resistant chronic myeloid leukemia in blast crisis, drug resistant Burkitt's lymphoma and drug resistant multiple myeloma, by administering an effective amount of a histone deactylase inhibitor of formula (I), either alone or in combination with a proteasome inhibitor, to a subject, e.g. a mammal (and more particularly a human) in need of such treatment. The present invention is particularly applicable to the treatment of drug resistant multiple myeloma, more particular to multiple myeloma resistant to proteasome inhibitors, even more particular to the treatment of bortezomib resistant multiple myeloma.

The term “drug resistant multiple myeloma” includes but is not limited to multiple myeloma resistant to one or more drugs selected from the group of thalidomide, dexamethasone, revlimid, doxorubicin, vincristine, cyclophosphamide, pamidronate, melphalan, defibrotide, prednisone, darinaparsin, belinostat, vorinostat, PD 0332991, LBH589, LAQ824, MGCD0103, HuLuc63, AZD 6244, Pazopanib, P276-00, plitidepsin, bendamustine, tanespimycin, enzastaurin, perifosine, ABT-737 or RAD001. The term “drug resistant multiple myeloma” also includes relapsed or refractory multiple myeloma.

With the term “drug resistant” is meant a condition which demonstrates intrinsic resistance or acquired resistance. With “intrinsic resistance” is meant the characteristic expression profile in cancer cells of key genes in relevant pathways, including but not limited to apoptosis, cell progression and DNA repair, which contributes to the more rapid growth ability of cancerous cells when compared to their normal counterparts. With “acquired resistance” is meant a multifactorial phenomenon occurring in tumor formation and progression that can influence the sensitivity of cancer cells to a drug. Acquired resistance may be due to several mechanisms such as but not limited to; alterations in drug-targets, decreased drug accumulation, alteration of intracellular drug distribution, reduced drug-target interaction, increased detoxification response, cell-cycle deregulation, increased damaged-DNA repair, and reduced apoptotic response. Several of said mechanisms can occur simultaneously and/or may interact with each other. Their activation and/or inactivation can be due to genetic or epigenetic events or to the presence of oncoviral proteins. Acquired resistance can occur to individual drugs but can also occur more broadly to many different drugs with different chemical structures and different mechanisms of action. This form of resistance is called multidrug resistance.

The combination according to the invention may be used for other therapeutic purposes,

for example:
a) the sensitisation of tumours to radiotherapy by administering the compound according to the invention before, during or after irradiation of the tumour for treating cancer;
b) treating arthropathies and osteopathological conditions such as rheumatoid arthritis, osteoarthritis, juvenile arthritis, gout, polyarthritis, psoriatic arthritis, ankylosing spondylitis and systemic lupus erythematosus;
c) inhibiting smooth muscle cell proliferation including vascular proliferative disorders, atherosclerosis and restenosis;
d) treating inflammatory conditions and dermal conditions such as ulcerative colitis, Crohn's disease, allergic rhinitis, graft vs. host disease, conjunctivitis, asthma, ARDS, Behcets disease, transplant rejection, uticaria, allergic dermatitis, alopecia greata, scleroderma, exanthema, eczema, dermatomyositis, acne, diabetes, systemic lupus erythematosis, Kawasaki's disease, multiple sclerosis, emphysema, cystic fibrosis and chronic bronchitis;
e) treating endometriosis, uterine fibroids, dysfunctional uterine bleeding and endometrial hyperplasia;
f) treating ocular vascularisation including vasculopathy affecting retinal and choroidal vessels;
g) treating a cardiac dysfunction;
h) inhibiting immunosuppressive conditions such as the treatment of HIV infections;
i) treating renal dysfunction;
j) suppressing endocrine disorders;
k) inhibiting dysfunction of gluconeogenesis;
l) treating a neuropathology for example Parkinson's disease or a neuropathology that results in a cognitive disorder, for example, Alzheimer's disease or polyglutamine related neuronal diseases;
m) treating psychiatric disorders for example schizophrenia, bipolar disorder, depression, anxiety and psychosis;
n) inhibiting a neuromuscular pathology, for example, amylotrophic lateral sclerosis;
o) treating spinal muscular atrophy;
p) treating other pathologic conditions amenable to treatment by potentiating expression of a gene;
q) enhancing gene therapy;
r) inhibiting adipogenesis;
s) treating parasitosis such as malaria.

Hence, the present invention discloses the above described combinations for use as a medicine as well as the use of a HDAC inhibitor of formula (I) with combined activity on class-I and class-IIb HDACs, either alone or in combination with a proteasome inhibitor, for the manufacture of a medicament for treating one or more of the above mentioned conditions.

Thus, the present invention discloses the use of a HDAC inhibitor of formula (I) with combined activity on class-I and class-IIb HDACs, either alone or in combination, for the manufacture of a medicament for the treatment of acute lymphoblastic leukemia, acute myelogenous leukemia, acute promyelocytic leukemia, acute myeloid leukemia, acute monocytic leukemia, lymphoma, chronic B cell leukemia, chronic myeloid leukemia, chronic myeloid leukemia in blast crisis, Burkitt's lymphoma and multiple myeloma.

The presents invention also discloses the use of a HDAC inhibitor of formula (I) with combined activity on class-I and class-IIb HDACs, either alone or in combination, for the manufacture of a medicament for the treatment of drug resistant tumors, such as but not limited to, hematopoietic tumors of lymphoid lineage e.g. drug resistant acute lymphoblastic leukemia, drug resistant acute myelogenous leukemia, drug resistant acute promyelocytic leukemia, drug resistant acute myeloid leukemia, drug resistant acute monocytic leukemia, drug resistant lymphoma, drug resistant chronic B cell leukemia, drug resistant chronic myeloid leukemia, drug resistant chronic myeloid leukemia in blast crisis, drug resistant Burkitt's lymphoma and drug resistant multiple myeloma.

The present invention further discloses the use of a HDAC inhibitor of formula (I) with combined activity on class-I and class-IIb HDACs, either alone or in combination, for the manufacture of a medicament for the treatment of drug resistant multiple myeloma, more in particular of multiple myeloma resistant to proteasome inhibitors, even more in particular of bortezomib resistant multiple myeloma.

The proteasome inhibitor and the HDAC inhibitor of formula (I) may be administered simultaneously (e.g. in separate or unitary compositions) or sequentially in either order. In the latter case, the two compounds will be administered within a period and in an amount and manner that is sufficient to ensure that an advantageous or synergistic effect is achieved. It will be appreciated that the preferred method and order of administration and the respective dosage amounts and regimes for each component of the combination will depend on the particular proteasome inhibitor and the HDAC inhibitor being administered, the route of administration of the combination, the particular tumor being treated and the particular host being treated. The optimum method and order of administration and the dosage amounts and regime can be readily determined by those skilled in the art using conventional methods and in view of the information set out herein.

The present invention further relates to a product containing as first active ingredient a HDAC inhibitor of formula (I) and as second active ingredient a proteasome inhibitor, as a combined preparation for simultaneous, separate or sequential use in the treatment of patients suffering from cancer.

Those skilled in the art could easily determine the effective amount from the test results presented hereinafter. In general it is contemplated that a therapeutically effective amount of a compound of formula (I) and of a proteasome inhibitor would be from 0.005 mg/kg to 100 mg/kg body weight, and in particular from 0.005 mg/kg to 10 mg/kg body weight. It may be appropriate to administer the required dose as two, three, four or more sub-doses at appropriate intervals throughout the day. Said sub-doses may be formulated as unit dosage forms, for example, containing 0.5 to 500 mg, and in particular 10 mg to 500 mg of active ingredient per unit dosage form.

In view of their useful pharmacological properties, the components of the combinations according to the invention, i.e. the proteasome inhibitor and the HDAC inhibitor may be formulated into various pharmaceutical forms for administration purposes. The components may be formulated separately in individual pharmaceutical compositions or in a unitary pharmaceutical composition containing both components. HDAC inhibitors can be prepared and formulated into pharmaceutical compositions by methods known in the art and in particular according to the methods described in the published patent specification mentioned herein and incorporated by reference.

The present invention therefore also relates to a pharmaceutical composition comprising a proteasome inhibitor and a HDAC inhibitor of formula (I) together with one or more pharmaceutical carriers. To prepare pharmaceutical compositions for use in accordance with the invention, an effective amount of a particular compound, in base or acid addition salt form, as the active ingredient is combined in intimate admixture with a pharmaceutically acceptable carrier, which carrier may take a wide variety of forms depending on the form of preparation desired for administration. These pharmaceutical compositions are desirably in unitary dosage form suitable, preferably, for administration orally, rectally, percutaneously, or by parenteral injection. For example, in preparing the compositions in oral dosage form, any of the usual pharmaceutical media may be employed, such as, for example, water, glycols, oils, alcohols and the like in the case of oral liquid preparations such as suspensions, syrups, elixirs and solutions; or solid carriers such as starches, sugars, kaolin, lubricants, binders, disintegrating agents and the like in the case of powders, pills, capsules and tablets. Because of their ease in administration, tablets and capsules represent the most advantageous oral dosage unit form, in which case solid pharmaceutical carriers are obviously employed. For parenteral compositions, the carrier will usually comprise sterile water, at least in large part, though other ingredients, to aid solubility for example, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. In the compositions suitable for percutaneous administration, the carrier optionally comprises a penetration enhancing agent and/or a suitable wetting agent, optionally combined with suitable additives of any nature in minor proportions, which additives do not cause a significant deleterious effect to the skin. Said additives may facilitate the administration to the skin and/or may be helpful for preparing the desired compositions. These compositions may be administered in various ways, e.g., as a transdermal patch, as a spot-on, as an ointment.

It is especially advantageous to formulate the aforementioned pharmaceutical compositions in dosage unit form for ease of administration and uniformity of dosage. Dosage unit form as used in the specification and claims herein refers to physically discrete units suitable as unitary dosages, each unit containing a predetermined quantity of active ingredient calculated to produce the desired therapeutic effect in association with the required pharmaceutical carrier. Examples of such dosage unit forms are tablets (including scored or coated tablets), capsules, pills, powder packets, wafers, injectable solutions or suspensions, teaspoonfuls, tablespoonfuls and the like, and segregated multiples thereof.

It may be appropriate to administer the required dose of each component of the combination as two, three, four or more sub-doses at appropriate intervals throughout the course of treatment. The sub-doses may be formulated as unit dosage forms, for example, in each case containing independently 0.01 to 500 mg, for example 0.1 to 200 mg and in particular 1 to 100 mg of each active ingredient per unit dosage form.

The term “the induction of acetylation of histones or other proteins” means the induction of the acetylation status of HDAC substrates such as but not limited to histones, e.g. histone 3, histone 4 and the like; tubulin, e.g. alpha-tubulin and the like; heat shock proteins, e.g. Hsp 90 and the like.

The term “the induction of proteins functionally regulated by said acetylation” means secondary effects such as but not limited to induction of Hsp70, induction of p21 and the like.

The invention also relates to a method for the characterisation of a HDAC inhibitor of formula (I) either alone or in combination with a proteasome inhibitor comprising the determination in a sample, of the amount of induction of acetylation of histones or other proteins, or of the induction of proteins functionally regulated by said acetylation. More in particular, the invention relates to a method for the characterisation of a HDAC inhibitor of formula (I) either alone or in combination with a proteasome inhibitor, comprising the determination in a sample of the amount of
a) induction of acetylation of histone 3, induction of acetylation of histone 4, or induction of p21 and
b) induction of acetylation of alpha-tubulin, induction of acetylation of Hsp 90, or induction of Hsp 70.

Most particular the invention relates to the above method, wherein the concentration needed to obtain induction under a) is in the same range as the concentration to obtain induction under b).

The determination in a sample of the amount of induction of acetylation of histones or other proteins or of the induction of proteins functionally regulated by said acetylation may encompass the identification of patients that respond to a treatment and thus may have a beneficial effect for the treatment of human cancer.

The determination in a sample of the amount of induction of acetylation of histones or other proteins or of the induction of proteins functionally regulated by said acetylation may encompass monitoring efficacy of a treatment in patients and thus may have a beneficial effect for the treatment of human cancer.

The determination in a sample of the amount of induction of acetylation of histones or other proteins or of the induction of proteins functionally regulated by said acetylation may encompass predicting therapeutic responses to a treatment and thus may have a beneficial effect for the treatment of human cancer.

Hence the present invention also relates to the use of a HDAC inhibitor of formula (I), with combined activity on class-I and class-IIb HDACs, either alone or in combination with a proteasome inhibitor, wherein the induction of hyperacetylation of histones or other proteins or the induction of proteins functionally regulated by said acetylation has a beneficial effect for the treatment of human cancer.

The sample may be derived from cells which have been treated with said HDAC inhibitor or said combination. The sample may also be derived from tissue affected by a disorder and/or from individuals treated with a HDAC inhibitor of formula (I) or a combination of a proteasome inhibitor and a HDAC inhibitor of formula (I)

The cells may be culture cells which have been contacted with said HDAC inhibitor or said combination. Said inhibitor or said combination can be added to the growth medium of the cells.

The cells may also be derived from a tissue and/or from an individual that was treated with said inhibitor or said combination.

Preferably, the method of characterization comprises only steps which are carried out in vitro. Therefore, according to this embodiment the step of obtaining the tissue material from the human or animal body is not encompassed by the present invention.

The cells are usually processed to be in a condition which is suitable for the method employed, for determining the induction of acetylation of histones or other proteins or the induction of proteins functionally regulated by said acetylation. Processing may include homogenization, extraction, fixation, washing and/or permeabilisation. The way of processing largely depends on the method used for the determination of the induction of acetylation of histones or other proteins or the induction of proteins functionally regulated by said acetylation. The sample may be derived from a biopsy of the patent. The biopsy may be further treated to yield a sample which is in a condition suitable for the method used for determining the induction of acetylation of histones or other proteins or the induction of proteins functionally regulated by said acetylation.

The amount of acetylation of proteins or the amount of induced protein may be determined by use of an antibody.

As used herein, the term “antibody” designates an immunoglobulin or a derivative thereof having the same binding specificity. The antibody used according to the invention may be a monoclonal antibody or an antibody derived from or comprised in a polyclonal antiserum. The term “antibody” further means derivatives such as Fab, F(ab′)2, Fv or scFv fragments. The antibody or the derivative thereof may be of natural origin or may be (semi)synthetically produced.

Western blotting may be used which is generally known in the art. The cellular material or tissue may be homogenized and treated with denaturing and/or reducing agents to obtain the samples. The sample may be loaded on a polyacrylamide gel to separate the proteins followed by transfer to a membrane or directly be spotted on a solid phase. The antibody is then contacted with the sample. After one or more washing steps the bound antibody is detected using techniques which are known in the art.

Immunohistochemistry may be used after fixation and permeabilisation of tissue material, e.g. slices of solid tumors, the antibody is then incubated with the sample, and following one or more washing steps the bound antibody is detected.

The amount of the induction of acetylation of histones or other proteins or the induction of proteins functionally regulated by said acetylation may be determined by ELISA. A variety of formats of the ELISA can be envisaged. In one format, the antibody is immobilized on a solid phase such as a microtiter plate, followed by blocking of aspecific binding sites and incubation with the sample. In another format, the sample is first contacted with the solid phase to immobilize the acetylated and/or induced proteins contained in the sample. After blocking and optionally washing, the antibody is contacted with the immobilized sample.

The amount of the induction of acetylation of histones or other proteins or the induction of proteins functionally regulated by said acetylation may be determined by flow cytometry. Cells, e.g. cell culture cells or blood cells or cells from bone marrow, are fixed and permeabilised to allow the antibody to reach the acetylated and/or induced proteins. After optional washing and blocking steps the antibody is contacted with the cells. Flow cytometry is then performed in accordance with procedures known in the art in order to determine cells having antibody bound to the acetylated and/or induced proteins.

To determine whether a HDAC inhibitor or a combination of a proteasome inhibitor and a HDAC inhibitor of formula (I) has his activity, one may determine the amount of acetylation of a protein or induction of a protein in a reference sample wherein the reference sample is derived from cells which have not been treated with said HDAC inhibitor or said combination. The determination of the amount of acetylation of proteins and/or the amount of induced protein in the sample and the reference sample may be performed in parallel. In the case of cell culture cells, two cellular compositions are provided, one of which is treated with said HDAC inhibitor or said combination whereas the other is left untreated. Subsequently both compositions are further processed and the respective amounts of acetylation of proteins and/or the amount of induced protein are determined. Alternatively, to determine whether a HDAC inhibitor or a combination of a proteasome inhibitor and a HDAC inhibitor of formula (I) has his activity, one may determine inhibition of cell proliferation.

In the case of patients, the sample is derived from a patient which has been treated with the HDAC inhibitor of formula (I) or the combination of a proteasome inhibitor and a HDAC inhibitor of formula (I). The reference sample is derived from another patient suffering from the same disorder who has not been treated with said HDAC inhibitor or said combination or from a healthy individual. The tissue from which the reference sample is derived corresponds to the tissue from which the sample is derived. For example, if the sample is derived from tumor tissue from a breast cancer patient the reference sample is also derived from tumor tissue from a breast cancer patient or from breast tissue from a healthy individual. It may also be envisaged that the sample and the reference sample are derived from the same individual. In this case, the tissue, from which the reference sample is derived was obtained from the individual prior to or after treatment of the individual with said HDAC inhibitor or said combination. Preferably, the tissue is obtained prior to the treatment to exclude possible after-effects of the inhibitor treatment after discontinuation of the treatment.

### Experimental Part

### A. Pharmacological Example

For the Cellular activity of the compounds of formula (I) which was determined on A2780 tumour cells using a colorimetric assay for cell toxicity or survival (Mosmann Tim, Journal of Immunological Methods 65: 55-63, 1983), reference is made to the experimental part of WO 2006/010750

The antiproliferative effects of HDAC inhibitors has been linked to the inhibition of class 1 HDACs, which consists of HDAC family members 1-3 and 8. The activity of JNJ 26481585 on HDAC 1 immuno-precipitated from A2780 cells and its potency when compared with R306465, SAHA, LBH-589 and LAQ-824 can be found in example A.1. The activity of JNJ 26481585 on HDAC 8 human recombinant enzyme and its potency when compared with R306465, SAHA, LBH-589 and LAQ-824 can be found in example A.2.

It was further investigated whether R306465 modulates the acetylation status of HDAC 1 substrates Histone 3 (H3) and Histone 4 (H4). Also the induction of cyclin dependent kinase inhibitor p21^{waf1,cip1} in A2780 ovarian carcinoma cells was investigated. P21^{waf1,cip1} is repressed as a consequence of histone acetylation, and plays a key role in the induction of cell cycle arrest in response to HDAC inhibitors (see example A.3.).

In order to assess the inhibition of HDAC 6, and the relative potency of the compounds for HDAC 1 versus HDAC 6, the acetylation of its substrate tubulin, and the induction of Hsp 70, which is the consequence of Hsp 90 acetylation, was monitored (see example A.4.).

### EXAMPLE A

### Class-I Specificity and Acetylation Effects of the Compounds of Formula (I)

### EXAMPLE A. 1

### Inhibition of HDAC 1 Enzyme Immuno-Precipitated from A2780 Cells

For HDAC 1 activity assays, HDAC 1 was immunoprecipitated from A2780 cell lysates and incubated with a concentration curve of the indicated HDAC inhibitor, and with a [³H]acetyl-labeled fragment of H4 peptide (50.000 cpm) [biotin-(6-aminohexanoic)Gly-Ala-(acetyl[³H]Lys-Arg-His-Arg-Lys-Val-NH₂](Amersham Pharmacia Biotech, Piscataway, N.J.). HDAC activity was assessed measuring release of free acetyl groups. Results are expressed as average IC₅₀ values±SD for three independent experiments.

| | HDAC 1 inhibition IC₅₀ in nM |
|---|---|
| JNJ 26481585 | 0.16 ± 0.02 |
| R306465 | 3.31 ± 0.78 |
| SAHA | 73 ± 26 |
| LAQ-824 | 0.29 ± 0.05 |
| LBH-589 | 0.23 ± 0.06 |

### EXAMPLE A.2

### Inhibition of HDAC 8 Human Recombinant Enzyme

For the inhibition of human recombinant HDAC 8, the HDAC 8 Colorimetric/Flourimetric Activity Assay/Drug Discovery Kit (Biomol; Cat. nr. AK-508) was used. Results are expressed as average IC₅₀ values (nM)±SD for three independent experiments. Assays were performed in duplicate and the standard error of the IC₅₀ was calculated using Graphpad Prism (Graphpad Software).

| | HDAC 8 inhibition IC₅₀ in nM |
|---|---|
| JNJ 26481585 | 34 ± 41 |
| R306465 | 23 ± 17 |
| SAHA | 370 ± 314 |
| LAQ-824 | 37 ± 23 |
| LBH-589 | 283 ± 29 |

### EXAMPLE A.3

### Acetylation of cellular HDAC 1 substrates and induction of p21^{waf1,cip1}

Human A2780 ovarian carcinoma cells were incubated with 0, 1, 3, 10, 30, 100, 300, 1000 and 3000 nM of the compounds for 24 h.

Total cell lysates were prepared and analysed by SDS-PAGE. Levels of acetylated H3 and H4 histones, total level of H3 proteins and levels p21^{waf1,cip1} protein were detected using rabbit polyclonal and mouse monoclonal antibodies, followed by enhanced chemoluminescence (ECL) detection.

Levels of acetylated H3 and H4 were detected with antibodies from Upstate Biotechnology (Cat. nr. 06-299 and 06-866), total level of H3 proteins was detected with antibodies from Abcam (Cat. nr. ab1791) and level of p21^{waf1,cip1} protein was detected with antibodies from Transduction Laboratories (Cat. nr. C24420). Appropriate dilutions of antibodies were incubated for either 1-2 h at room temperature or overnight at 4° C. In order to control for equal loading, blots were stripped and re-probed with mouse monoclonal anti-actin IgM (Ab-1, Oncogene Research Products). in order to control the efficiency of extraction of nuclear proteins blots were stripped and re-probed with and with anti-lamin B1 (Zymed; Cat. nr. 33.2000). Protein-antibody complexes were then visualized by chemiluminescence (Pierce Chemical Co) or fluorescence (Odyssey) according to the manufacturer's instructions. The experiments were performed three times.

| | concentration (nM) when induction of |
|---|---|
| | acetylation of histone H3 and H4 and |
| | induction of p21^{waf1, cip1} is observed |
| JNJ 26481585 | 10 |
| R306465 | 100 |
| SAHA | 3000 |
| LAQ-824 | 10 |
| LBH-589 | 10 |

### EXAMPLE A.4

### Acetylation of Tubulin and the Induction of Hsp 70

Human A2780 ovarian carcinoma cells were incubated with 0, 1, 3, 10, 30, 100, 300, 1000 and 3000 nM of the compounds for 24 h.

Total cell lysates were prepared and analysed by SDS-PAGE. Levels of total and acetylated tubulin were detected using antibodies from Sigma: clones DM1A (Cat. nr. T9026) and 6-111B (Cat. nr. T6793). Hsp 70 protein was detected with an antibody from Stressgen (Cat. nr. SPA-810), followed by ECL detection. Appropriate dilutions of antibodies were incubated for either 1-2 h at room temperature or overnight at 4° C. In order to control for equal loading, blots were stripped and re-probed with mouse monoclonal anti-actin IgM (Ab-1, Oncogene Research Products). In order to control the efficiency of extraction of nuclear proteins blots were stripped and re-probed with and with anti-lamin B1 (Zymed; Cat. nr. 33.2000). Protein-antibody complexes were then visualized by chemiluminescence (Pierce Chemical Co) or fluorescence (Odyssey) according to the manufacturer's instructions. The experiments were performed three times.

| | concentration (nM) when start of |
|---|---|
| | induction of acetylation of tubulin and |
| | induction of Hsp 70 is observed |
| JNJ 26481585 | 30 |
| R306465 | 1000 |
| SAHA | 100 |
| LAQ-824 | 30 |
| LBH-589 | 30 |

### EXAMPLE B

### Inhibition of Human Hematological Tumor Cell Proliferation

Evaluation of anti-proliferative activity of JNJ 26481585 in a pannel of human hematological tumor cell lines was outsourced at Oncodesign (Dijon, France). Tumor cells were grown as cell suspension in the corresponding appropriate culture medium at 37° C. in a humidified 5% CO₂ incubator. Mycoplasma-free tumor cells were seeded in 96-well flat-bottom microtitration plates and incubated at 37° C. for 24 hr in culture medium containing 10% FCS. Tumor cells were then exposed to vehicle (control) or increasing concentrations of JNJ 26481585 (5 different concentrations*), Bortezomib (5 different concentrations*), or combination of both drugs at various ratio. Cells were then incubated for an additional 72 hr. The cytotoxic activity of the compound(s) was revealed by standard MTS assay by measurement of absorbency at 490 nm. The compound interactions (synergy, additivity or antagonism) was calculated by multiple drug effect analysis and was performed by the median equation principle according to the methodology described by Chou & Talalay [CHOU et al. (1984) Adv. Enzyme Regul. 22: 27-55; CHOU et al. (1991) in Encyclopaedia of human Biology. Academic Press. 2: 371-379; CHOU et al. (1991) in Synergism and antagonism in chemotherapy. Academic Press: 61-102; CHOU et al. (1994) J. Natl. Cancer Inst. 86: 1517-1524] *: based on pre-determination of anti-proliferative activity of each drug used as single agent, concentrations were chosen not to exceed 50% inhibition in each of the selected cell lines.

### EXAMPLE B.1

### Inhibition of Human Hematological Tumor Cell Proliferation by JNJ 26481585

TABLE F.1
Results are expressed as the mean IC₄₀ value (i.e., concentration,
expressed in nM, required to reach 40% inhibition of cell proliferation) ±
SD, determined from 3 independent reliable experiments.

| Cell line | Type | Mean | SD |
|---|---|---|---|
| CCRF-CEM | Acute lymphoblastic leukemia | 11.93 | 7.18 |
| Jurkat clone E6-1 | Acute lymphoblastic leukemia | 9.22 | 1.78 |
| KG-1 | Acute myelogenous leukemia | 13.39 | 10.48 |
| MOLT-4 | Acute lymphoblastic leukemia | 42.96 | 56.25 |
| SUP-B15 | Acute lymphoblastic leukemia | 1.13 | |
| HL-60 | Acute promyelocytic leukemia | 24.28 | 10.72 |
| OCI-AML2 | Acute myeloid leukemia | 19.56 | 13.93 |
| THP-1 | Acute monocytic leukemia | 50.65 | 22.84 |
| EHEB | Chronic B cell leukemia | 165.81 | 128.15 |
| BV-173 | Chronic B cell leukemia | 4.54 | 4.28 |
| K-562 | Chronic myeloid leukemia | 15.91 | 8.29 |
| KCL-22 | Chronic myeloid leukemia | 7.71 | 5.14 |
| LAMA-84 | Chronic myeloid leukemia in | 30.40 | 19.93 |
| | blast crisis | | |
| U-937 | Lymphoma | 23.67 | 10.14 |
| Daudi | Burkitt's lymphoma | 9.08 | 1.27 |
| Namalwa | Burkitt's lymphoma | 4.65 | 4.32 |
| Raji | Burkitt's lymphoma | 26.34 | 16.11 |
| Ramos | Burkitt's lymphoma | 4.66 | 2.82 |
| ARH-77 | Myeloma | 40.42 | 24.32 |
| RPMI 8226 | Myeloma | 6.41 | 5.44 |

### EXAMPLE B.2

### Inhibition of Human Hematological Tumor Cell Proliferation by Bortezomib

TABLE F.2
Results are expressed as the mean IC₄₀ value (i.e., concentration,
expressed in nM, required to reach 40% inhibition of cell proliferation) ±
SD, determined from 3 independent reliable experiments.

| Cell line | Type | Mean | SD |
|---|---|---|---|
| CCRF-CEM | Acute lymphoblastic leukemia | 4.40 | 0.84 |
| Jurkat clone E6-1 | Acute lymphoblastic leukemia | 5.63 | 2.68 |
| KG-1 | Acute myelogenous leukemia | 3.36 | 0.83 |
| MOLT-4 | Acute lymphoblastic leukemia | 12.14 | 12.91 |
| SUP-B15 | Acute lymphoblastic leukemia | 2.40 | |
| HL-60 | Acute promyelocytic leukemia | 13.38 | 1.99 |
| OCI-AML2 | Acute myeloid leukemia | 11.64 | 11.61 |
| THP-1 | Acute monocytic leukemia | 5.83 | 0.94 |
| EHEB | Chronic B cell leukemia | 6.02 | 0.34 |
| BV-173 | Chronic B cell leukemia | 2.77 | 0.20 |
| K-562 | Chronic myeloid leukemia | 12.83 | 4.11 |
| KCL-22 | Chronic myeloid leukemia | 1.74 | 1.56 |
| LAMA-84 | Chronic myeloid leukemia in | 2.61 | 0.46 |
| | blast crisis | | |
| U-937 | Lymphoma | 5.68 | 1.07 |
| Daudi | Burkitt's lymphoma | 2.68 | 0.54 |
| Namalwa | Burkitt's lymphoma | 4.48 | 1.00 |
| Raji | Burkitt's lymphoma | 5.20 | 0.69 |
| Ramos | Burkitt's lymphoma | 1.83 | 0.10 |
| ARH-77 | Myeloma | 7.21 | 2.22 |
| RPMI 8226 | Myeloma | 4.23 | 0.99 |

### EXAMPLE B.3

### Inhibition of Human Hematological Tumor Cell Proliferation by JNJ 26481585 in combination with Bortezomib

TABLE 3

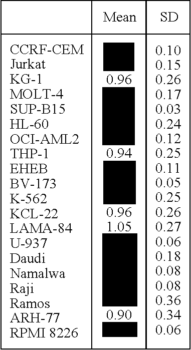

Results are expressed as the mean Combination Index (CI ± SD) of median CI values in each individual studies (3 independent reliable experiments) and calculated from each individual combination ratio. CI lower than 0.9 indicates ‘Synergy’ (grey) aand CI between 0.91 and 1.09 indicates ‘Additivity’ (white).

1. A combination of a proteasome inhibitor and a histone deacetylase inhibitor of formula (I) 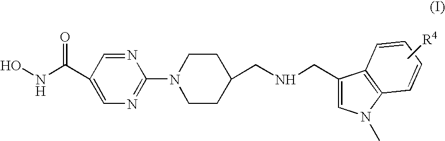
the pharmaceutically acceptable acid or base addition salts and the stereochemically isomeric forms thereof, wherein
R⁴ is selected from hydrogen or halo.

2. A combination as claimed in claim 1 wherein the histone deacetylase inhibitor of formula (I) is selected from the following compounds:

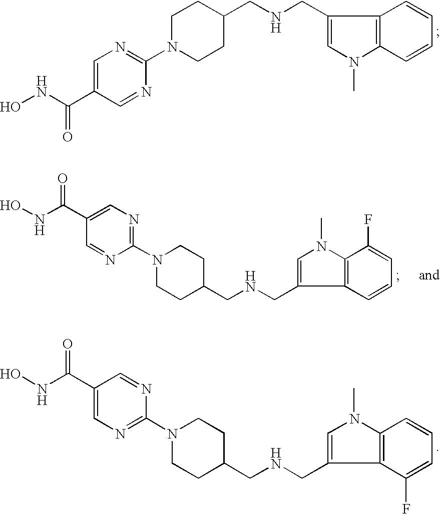

3. A combination as claimed in claim 2 wherein the histone deacetylase inhibitor of formula (I) is compound No. 1a:

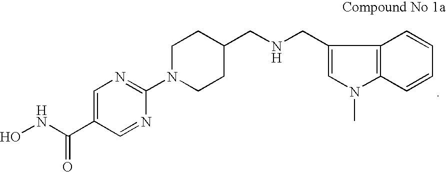

4. A combination as claimed in claim 1 wherein the proteasome inhibitor is bortezomib.

5. A combination as claimed in claim 1 in the form of a pharmaceutical composition comprising a proteasome inhibitor and a histone deacetylase inhibitor of formula (I) together with one or more pharmaceutical carriers.

6. A combination as claimed in claim 5 for simultaneous, separate or sequential use.

7. (canceled)

8. (canceled)

9. A method for the treatment of acute lymphoblastic leukemia, acute myelogenous leukemia, acute promyelocytic leukemia, acute myeloid leukemia, acute monocytic leukemia, lymphoma, chronic B cell leukemia, chronic myeloid leukemia, chronic myeloid leukemia in blast crisis, Burkitt's lymphoma and multiple myeloma in a subject in need of treatment, said method comprising administering a therapeutically effective amount of a histone deactylase inhibitor of formula (I): 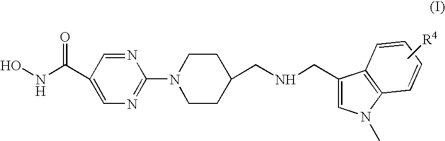
the pharmaceutically acceptable acid or base addition salts and the stereochemically isomeric forms thereof, wherein R⁴ is selected from hydrogen or halo, either alone or in combination with a proteasome inhibitor.

10. A method for the treatment of drug resistant acute lymphoblastic leukemia, drug resistant acute myelogenous leukemia, drug resistant acute promyelocytic leukemia, drug resistant acute myeloid leukemia, drug resistant acute monocytic leukemia, drug resistant lymphoma, drug resistant chronic B cell leukemia, drug resistant chronic myeloid leukemia, drug resistant chronic myeloid leukemia in blast crisis, drug resistant Burkitt's lymphoma and drug resistant multiple myeloma in a subject in need of treatment, said method comprising administering a therapeutically effective amount of a histone deactylase inhibitor of formula (I): 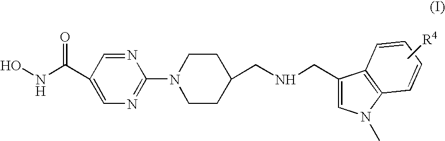
the pharmaceutically acceptable acid or base addition salts and the stereochemically isomeric forms thereof, wherein R⁴ is selected from hydrogen or halo, either alone or in combination with a proteasome inhibitor.

11. The method of claim 9, wherein said method is for treatment of bortezomib resistant multiple myeloma.

12. The method of claim 9, further comprising the induction of hyperacetylation of histones or the induction of proteins functionally regulated by said acetylation for a beneficial effect for the treatment of human cancer.

13. A method for the characterisation of a histone deacetylase inhibitor of formula (I) as defined in claim 1, either alone or in combination with a proteasome inhibitor, said method comprising the determination in a sample, of the amount of induction of acetylation of histones or other proteins, or of the induction of proteins functionally regulated by said acetylation.

14. A method for the characterisation of a histone deacetylase inhibitor of formula (I) as defined in claim 1, either alone or in combination with a proteasome inhibitor, said method comprising the determination in a sample of the amount of
a) induction of acetylation of histone 3, induction of acetylation of histone 4, or induction of p21 and
b) induction of acetylation of alpha-tubulin, induction of acetylation of Hsp 90, or induction of Hsp 70.

15. A combination as claimed in claim 2 wherein the proteasome inhibitor is bortezomib.

16. A combination as claimed in claim 3 wherein the proteasome inhibitor is bortezomib.

17. A combination as claimed in claim 2 in the form of a pharmaceutical composition comprising a proteasome inhibitor and a histone deacetylase inhibitor of formula (I) together with one or more pharmaceutical carriers.

18. A combination as claimed in claim 3 in the form of a pharmaceutical composition comprising a proteasome inhibitor and a histone deacetylase inhibitor of formula (I) together with one or more pharmaceutical carriers.

19. A combination as claimed in claim 4 in the form of a pharmaceutical composition comprising a proteasome inhibitor and a histone deacetylase inhibitor of formula (I) together with one or more pharmaceutical carriers.

20. The method of claim 10, wherein said method is for treatment of bortezomib resistant multiple myeloma.
